(19) United States
(12) Patent Application Publication
(10) Pub. No.: US 2011/0239739 A1
(43) Pub. Date: Oct. 6, 2011

(54) METHOD OF MANUFACTURING GAS SENSOR, AND GAS SENSOR

(75) Inventors: Hidekazu Katou, Aichi (JP); Masataka Taguchi, Aichi (JP); Yasuhiro Fujita, Gifu (JP); Takayoshi Atsumi, Aichi (JP)

(73) Assignee: NGK SPARK PLUG CO., LTD., Nagoya-shi, Aichi (JP)

(21) Appl. No.: 13/133,281

(22) PCT Filed: Feb. 22, 2010
(86) PCT No.: PCT/JP2010/001119

§ 371 Date: Jun. 7, 2011

(30) Foreign Application Priority Data
Mar. 2, 2009 (JP) 2009-047562

(51) Int. Cl. G01N 33/00; B23K 26/28

(52) U.S. Cl. 73/31.05; 219/121.64

(57) ABSTRACT

The present invention relates to improvement in the corrosion resistance of a weld joint between a metal shell and an outer casing. As a solution to this object, a metal shell 11 and an outer metal casing 16 are laser welded to each other via a weld joint 100 by irradiating a laser beam onto the entire circumference of a crimped area 90 in a circumferential direction of the outer metal casing 16. This laser welding operation is performed at a plurality of positions (two positions) displaced from each other in the direction of an axis O so that the weld joint 100 is formed with a front weld zone 110 and a rear weld zone 120.

### TECHNICAL FIELD

The present invention relates to a method of manufacturing a gas sensor, which is used to measure the concentration of a specific gas component in a measurement gas, and to a gas sensor.

### BACKGROUND ART

There is known a gas sensor, which includes an metal shell, a sensor element retained in the metal shell, with a sensing section thereof being exposed to a measurement gas, to measure the concentration of a specific gas component in the measurement gas, and an outer casing joined to a rear end portion of the metal shell. In the manufacturing of this type of gas sensor, the metal shell and the outer casing are generally joined by laser welding. More specifically, the joining of the metal shell and the outer casing is done by inserting the rear end portion of the metal shell into a front end portion of the outer casing, crimping an overlap area between the metal shell and the outer casing for temporary fixing of the outer casing onto the metal shell, and then, irradiating a laser beam onto the entire circumference of the crimped area from outside the outer casing.

### PRIOR ART DOCUMENTS

### Patent Document

Patent Document 1: Japanese Laid-Open Patent Publication No. 11-239888

### SUMMARY OF THE INVENTION

### Problems to be Solved by the Invention

As the outer casing is crimped at a position apart from a front edge of the outer casing and as such a crimped area is subjected to laser welding to form a weld zone on the crimped area, there is a narrow gap left between an outer circumferential surface of the metal shell and an inner circumferential surface of the outer casing from the front edge of the outer casing to the weld zone. If the gas sensor gets wet during its use, water penetrates into the gap between the outer casing and the metal shell so that the weld zone may be held in contact with water for a long term. During the long-term contact of the weld zone and water, corrosion proceeds at an interface between the weld zone and non-weld zone in view of the fact that the interface of the weld zone, which has been once molten by laser welding, is relatively susceptible to corrosion by water. This results in a failure of the gas sensor due to the entry of water into the gas sensor.

The present invention has been made in view of the above circumstances. It is an object of the present invention to improve the corrosion resistance of a weld joint between a metal shell and an outer casing of a gas sensor.

### Means for Solving the Problems

[Application Aspect 1]

A manufacturing method of a gas sensor, the gas sensor comprising: a sensor element extending in an axis direction of the gas sensor and having at a front end portion thereof a sensing section to detect a measurement gas; a metal shell having a cylindrical portion to surround an outer circumference of the sensor element, with the front end portion and a rear end portion of the sensor element being exposed to an outside of the metal shell; and a cylindrical outer casing fixed to the metal shell to surround the rear end portion of the sensor element, the manufacturing method comprising: an outer casing placing step for placing the outer casing on the metal shell in such a manner that a front end portion of the outer casing surrounds the cylindrical portion of the metal shell; and a welding step for performing a laser welding operation on the entire circumference of an overlap area between the front end portion of the outer casing and the cylindrical portion of the metal shell and thereby forming a weld zone astride a boundary between the front end portion of the outer casing and the cylindrical portion of the metal shell, wherein, in the welding step, the laser welding operation is performed a plurality of times at positions displaced from each other in the axis direction of the gas sensor.

[Application Aspect 2]

The manufacturing method of the gas sensor according to Application Aspect 1, wherein the laser welding operation is performed the plurality of times to form a plurality of weld zones in the welding step in such a manner that adjacent two of the weld zones have inner weld regions located in the cylindrical portion of the metal shell and partially overlapping each other.

[Application Aspect 3]

The manufacturing method of the gas sensor according to Application Aspect 1 or 2, wherein the laser welding operation is performed the plurality of times by displacing the position of the laser welding operation from a front side to a rear side of the overlap area in the welding step.

[Application Aspect 4]

The manufacturing method of the gas sensor according to any one of Application Aspects 1 to 3, wherein the welding step is carried out to form a first weld zone by the first laser welding operation and to form a second weld zone by the second or subsequent laser welding operation in such a manner that the second weld zone is greater in depth than the first weld zone.

[Application Aspect 5]

A gas sensor, comprising: a sensor element extending in an axis direction of the gas sensor and having at a front end portion thereof a sensing section to detect a measurement gas; a metal shell having a cylindrical portion to surround an outer circumference of the sensor element, with the front end portion and a rear end portion of the sensor element being exposed to an outside of the metal shell; a cylindrical outer casing fixed to the metal shell to surround the rear end portion of the sensor element; and a plurality of weld zones each formed astride a boundary between a front end portion of the outer casing and the cylindrical portion of the metal shell throughout the entire circumference of an overlap area between the front end portion of the outer casing and the cylindrical portion of the metal shell and displaced in position from each other in the axis direction of the gas sensor.

[Application Aspect 6]

The gas sensor according to Application Aspect 5, wherein adjacent two of the weld zones partially overlap each other in such a manner that, when viewed in cross section along the axis, a rear one of the adjacent two of the weld zones partially overlays a front one of the adjacent two of the weld zones.

[Application Aspect 7]

The gas sensor according to Application Aspect 6, wherein the rear one of the adjacent two of the weld zones is greater in depth than the front one of the adjacent two of the weld zones.

[Application Aspect 8]

The gas sensor according to Application Aspect 5, wherein adjacent two of the weld zones have inner weld regions located in the cylindrical portion of the metal shell and partially overlapping each other.

[Application Aspect 9]

The gas sensor according to Application Aspect 5, wherein the weld zones includes a first weld zone and a second weld zone partially overlaying the first weld zone and having a depth greater than a width of the first weld zone.

[Application Aspect 10]

The gas sensor according to any one of Application Aspects 5 to 9, wherein the weld zones extend to a point not more than half of a thickness of the cylindrical portion of the metal shell.

### Effects of the Invention

In the gas sensor manufacturing method of Application Aspect 1, the laser welding operation is performed a plurality of times at positions axially displaced from each other on the overlap area between the outer casing and the metal shell. It is therefore possible to increase the overall axial width of the weld joint astride the boundary between the front end portion of the outer casing and the cylindrical portion of the metal shell (hereinafter referred to as “weld width”) and possible to retard the progress of corrosion of the weld joint and, by extension, to retard the entry of water into the gas sensor.

In the gas sensor manufacturing method of Application Aspect 2, the laser welding operation is performed the plurality of times to form a plurality of weld zones in such a manner that the inner weld regions of adjacent two of the weld zones partially overlap each other. It is possible to increase the weld strength of the outer casing and the metal shell by the formation of such an overlap between the inner weld regions.

If the laser welding operation is repeatedly performed by displacing the position of the laser welding operation from the rear side to the front side of the overlap area, the front end portion of the outer casing may be expanded radially outwardly every time the weld zone is formed by the laser welding operation. This results in an increase of the gap between the outer casing and the metal shell. As it becomes more likely that water will penetrate into such an increased gap, the progress of corrosion of the weld zone may be hastened. In particular, the front weld zone may not be provided in desired form due to the increase of the gap between the outer casing and the metal shell. In the gas sensor manufacturing method of Application Aspect 3, the laser welding operation is repeatedly performed by displacing the position of the laser welding operation from the front side to the rear side of the overlap area. It is thus possible to prevent the gap between the outer casing and the metal shell from being increased during the welding step and, as a result, possible to not only avoid the entry of water into the gas sensor but also attain the desired form of the weld zone.

The cylindrical portion of the metal shell and the front end portion of the outer casing have not yet been joined together before the first laser welding operation. This makes it difficult, despite the intention to form the weld zone with a great depth, to provide the weld zone in such desired form. In the gas sensor manufacturing method of Application Aspect 4, the laser welding operation is repeatedly preformed in such a manner that the depth of the first weld zone formed by the first laser welding operation becomes slightly smaller the target depth and in such a manner that the depth of the second weld zone formed by the second or subsequent laser welding operation becomes greater than the depth of the first weld zone. As the cylindrical portion of the metal shell and the front end portion of the outer casing have been joined via the first weld zone at the time of formation of the second weld zone by the second or subsequent welding operation, it is possible to attain the desired great depth form of the second weld zone.

In the gas sensor of Application Aspect 5, a plurality of weld zones are formed at positions axially displaced from each other on the overlap area between the outer casing and the metal shell. It is therefore possible to increase the weld width and possible to retard the progress of corrosion of the weld zones and, by extension, to retard the entry of water into the gas sensor.

In the gas sensor of Application Aspect 6, two adjacent weld zones are formed in such a manner that the rear one of the two adjacent weld zones partially overlays the front one of the two adjacent weld zones. In other words, the laser welding operation is repeatedly performed by displacing the position of the laser welding from the front side to the rear side of the overlap area. It is thus possible to prevent the gap between the outer casing and the metal shell from being increased during the welding step and, as a result, possible to not only avoid the entry of water into the gas sensor but also attain the desired form of the weld zone. Herein, the expression “the rear weld zone partially overlays the front weld zone” means that, when the gas sensor (weld zones) is viewed in cross section along the axis, there can be visually recognized a border of the rear weld zone but not cannot be visually recognized a border of the front weld zone in the overlap between the front and rear weld zones.

In the gas sensor of Application Aspect 7, the rear weld zone is made greater in depth than the front weld zone. It is possible to attain the desired form of the front weld zone by setting the depth of the front weld zone slightly smaller than the target depth. It is also possible to attain the desired form of the rear weld zone by setting the depth of the rear weld zone greater than the depth of the front weld zone.

In the gas sensor of Application Aspect 8, the weld zones are formed by repeating the laser welding operation in such a manner that the inner weld regions of the two adjacent weld zones partially overlap each other. It is thus possible to increase the weld strength of the outer casing and the metal shell.

In the gas sensor of Application Aspect 9, it is possible to, even though the first weld zone is formed in a state that the metal shell and the outer casing have not yet joined together, attain the desired form of the first weld zone by setting the depth of the first weld zone slightly smaller than the target depth. It is also possible to attain the desired form of the second weld zone by setting the depth of the second weld zone greater than the depth of the first weld zone. Herein, the expression “the second weld zone partially overlays the first weld zone” means that, when the gas sensor (weld zones) is viewed in cross section along the axis, there can be visually recognized a border of the second weld zone but not cannot be visually recognized a border of the first weld zone in the overlap between the first and second weld zones.

In the gas sensor of Application Aspect 10, the weld zones extend to a point not more than half of the thickness of the cylindrical portion of the metal shell. In this case, it is possible to increase the weld strength of the outer casing and the metal shell as the components of the outer casing and the metal shell can be molten and mixed together assuredly. If the weld zones exceed half of the thickness of the cylindrical portion of the metal shell, the weld strength of the outer casing and the metal shell may be decreased due to the deterioration of the balance of the mixing ratio of the components of the outer casing and the metal shell.

In the present invention, the above various aspects may be appropriately modified, combined or partially omitted according to the applications.

### BRIEF DESCRIPTION OF DRAWINGS

FIG. 1 is a section view of a gas sensor 10 according to a first embodiment of the present invention.

FIG. 2 is a schematic section view showing the detailed configuration of a weld joint 100 between a metal shell 11 and an outer metal casing 16 according to the first embodiment of the present invention.

FIG. 3 is a flowchart of process steps for manufacturing the gas sensor 10 according to the first embodiment of the present invention.

FIG. 4 is a schematic view showing a welding step during the manufacturing of the gas sensor 10 according to the first embodiment of the present invention.

FIG. 5 is a schematic section view showing the detailed configuration of a weld joint 100a between a metal shell 11 and an outer metal casing 16 according to a first modification example of the present invention.

FIG. 6 is a schematic section view showing the detailed configuration of a weld zone 110b between a metal shell 11 and an outer metal casing 16 according to a second modification example of the present invention.

### BEST MODES FOR CARRYING OUT THE INVENTION

### A. First Embodiment

A1. Gas Sensor Configuration

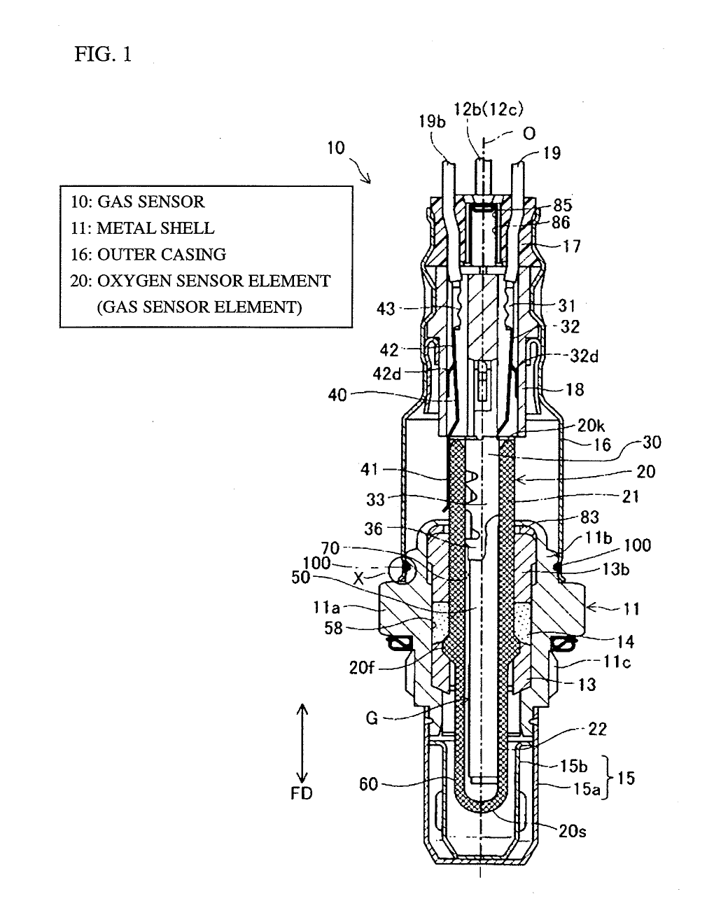

FIG. 1 is a section view of a gas sensor 10 according to a first embodiment of the present invention, which is designed as an oxygen sensor to detect oxygen in exhaust gas from an internal combustion engine. In general, the gas sensor 10 includes an oxygen sensor element 20, a metal shell 11, an outer metal casing 16, an inner terminal member 30, an outer terminal member 40 and a ceramic heater 50.

In FIG. 1, an axis O of the gas sensor 10 is indicated. Herein, the terms “front” and “rear” refer to sides of a structural member closer to a solid electrolyte body 21 and a grommet 17, respectively, with respect to a direction of the axis O (i.e. lower and upper sides of FIG. 1); and the term “longitudinal direction FD” refers to a direction in parallel to the direction of the axis O (i.e. a vertical direction of FIG. 1).

The oxygen sensor element 20 is formed into a bottomed cylindrical shape along the direction of the axis O (the vertical direction of FIG. 1). A front end 20s of the oxygen sensor element 20 (the upper side of FIG. 1) is closed, whereas a rear end 20k of the oxygen sensor element 20 (the lower side of FIG. 1) is open. The oxygen sensor element 20 includes a solid electrolyte body 21 having oxygen ion conductivity, an outer electrode 60 formed by e.g. plating on a part of an outer circumferential surface of the solid electrolyte body 21 and an inner electrode 70 formed by e.g. plating on a part of an inner circumferential surface of the solid electrolyte body 21. A sensing section 22 is provided on the oxygen sensor element 20 at a position close to the front end 20s. Further, an engagement flange portion 20f is provided on an outer circumference of the oxygen sensor element 20 at around a center position in the direction of the axis O for engagement with the metal shell 11 as explained below.

The metal shell 11 is formed into a cylindrical shape so as to surround a part of the outer circumference of the oxygen sensor element 20. An insulator 13 is retained in a through hole 58 of the metal shell 11 through a metal packing (not shown in the drawing) so that the engagement flange portion 20f is engaged with the insulator 13 via the metal packing. A talc 14, a sleeve 13b and a metal packing 83 are also arranged in through hole 58 of the metal shell 11 at a rear side of the insulator 13 so as to, by crimping a rear end of the metal shell 11, keep the oxygen sensor element 20 hermetically sealed in the metal shell 11.

A protector 15 is attached to a front end part of the metal shell 11 so as to surround and protect therewith the sensing section 22 of the oxygen sensor element 20 protruding from the front open end of the metal shell 11. In the first embodiment, the protector 15 has a double structure formed with an outer protector member 15a and an inner protector member 15b. A plurality of gas passage holes are made in the inner and outer protector members 15a and 15b for passage of the exhaust gas. The exhaust gas is thus fed to the outer electrode 60 of the oxygen sensor element 20 through the gas passage holes of the protector 15.

The metal shell 11 has, on an outer circumferential surface thereof, a hexagonal portion 11a and a thread portion 11c located at a front side of the hexagonal portion 11a. The metal shell 11 also has a cylindrical portion 11b located at a rear side of the hexagonal portion 11a and joined to a cylindrical front end portion 16a of the outer metal casing 16 by inserting the cylindrical portion 11b of the metal shell 11 in the front end portion 16a of the outer metal casing 16 and performing laser welding on these portions 16a and 11b from outside the outer metal casing 16. By such laser welding process, a weld joint 100 is formed between the metal shell 11 and the outer metal casing 16. The detailed configuration of the weld joint 100 between the metal shell 11 and the outer metal casing 16 will be explained later in detail. The outer metal casing 16 is formed into a cylindrical shape of stainless steel such as SUS304 and is attached to a rear end part of the metal shell 11 so as to surround and protect therewith the rear end portion of the oxygen sensor element 20 protruding from the rear open end of the metal shell 11 as well as to surround therewith a separator 18. A grommet 17 of fluoro rubber is fixed in an opening of a rear end of the outer metal casing 16 by crimping the rear end of the outer metal casing 16 to thereby close the opening of the rear end of the outer metal casing 16. The separator 18 is formed of an insulating alumina ceramic material and arranged in the outer metal casing 16 at a front side of the grommet 17. Further, sensor output leads 19 and 19b and heater leads 12b and 12c are passed through the grommet 17 and the separator 18. A through hole is made in the center of the grommet 17 along the direction of the axis O; and a metal pipe 86 is fitted in the though hole of the grommet 17 and covered with a water-repellent, air-permeable sheet-form filter 85. In this arrangement, the gas outside the gas sensor 10 is introduced into the outer metal casing 16 through the filter 85 and then introduced into an inner space G of the oxygen sensor element 20.

The outer terminal member 40 is formed of a stainless steel sheet and provided with an outer fitting portion 41, a separator insertion portion 42 and a connector portion 43. The separator insertion portion 42 is inserted in the separator 18. A separator contact section 42d is branched and protrudes from the separator insertion portion 42 so that the outer terminal member 40 is retained in the separator 18 by elastic contact of the separator contact section 42d with an inner wall of the separator 18.

The connector portion 43 is located at a rear side of the separator insertion portion 42 so as to hold a core wire of the sensor output lead 19b by crimping and thereby establish an electrical connection between the outer terminal member 40 and the sensor output lead 19b.

The outer fitting portion 41 is located at a front side of the separator insertion portion 42 so as to hold an outer circumference of the rear end portion of the oxygen sensor element 20 and thereby establish an electrical connection between the outer terminal member 40 and the outer electrode 60 of the oxygen sensor element 20. An electromotive force generated at the outer electrode 60 is outputted to the outside of the gas sensor 10 through the outer terminal member 40 and the sensor output lead 19b.

The inner terminal member 30 is formed of a stainless steel sheet and provided with an insertion portion 30, a separator insertion portion 32 and a connector portion 31. The separator insertion portion 32 is inserted in the separator 18. A separator contact section 32d is branched and protrudes from the separator insertion portion 32 so that the inner terminal member 30 is retained in the separator 18 by elastic contact of the separator contact section 32d with the inner wall of the separator 18.

The connector portion 31 is located at a rear side of the separator insertion portion 32 so as to hold a core wire of the sensor output lead 19 by crimping and thereby establish an electrical connection between the inner terminal member 30 and the sensor output lead 19.

The insertion portion 33 is located at a front side of the separator insertion portion 32. The insertion portion 33 is inserted in the oxygen sensor element 20 and brought, by its elastic force, into contact with and pressed against the inner electrode 70 on the inner circumference of the oxygen sensor element 20, so as to maintain electrical conduction between the inner terminal member 30 and the inner electrode 70 of the oxygen sensor element 20. An electromotive force generated at the inner electrode 70 is outputted to the outside of the gas sensor 10 through the inner terminal member 30 and the sensor output lead 19.

A heater pressing section 36 is provided on a front end of the insertion portion 33 so as to press a lateral surface of the ceramic heater 50 against the inner circumference of the oxygen sensor element 20.

The ceramic heater 50 is arranged in the inner space G of the oxygen sensor element 20 and held by the inner terminal member 30 to maintain its attitude. As connection terminals of the ceramic heater 50 are connected to the heater leads 12b and 12c, the ceramic heater 50 generates and applies heat to the inner circumferential surface of the solid electrolyte body 21 upon power supply through the heater leads 12b and 12c. The gas sensor 10 of the first embodiment is structured as mentioned above.

A2. Detailed Configuration of Weld Joint 100

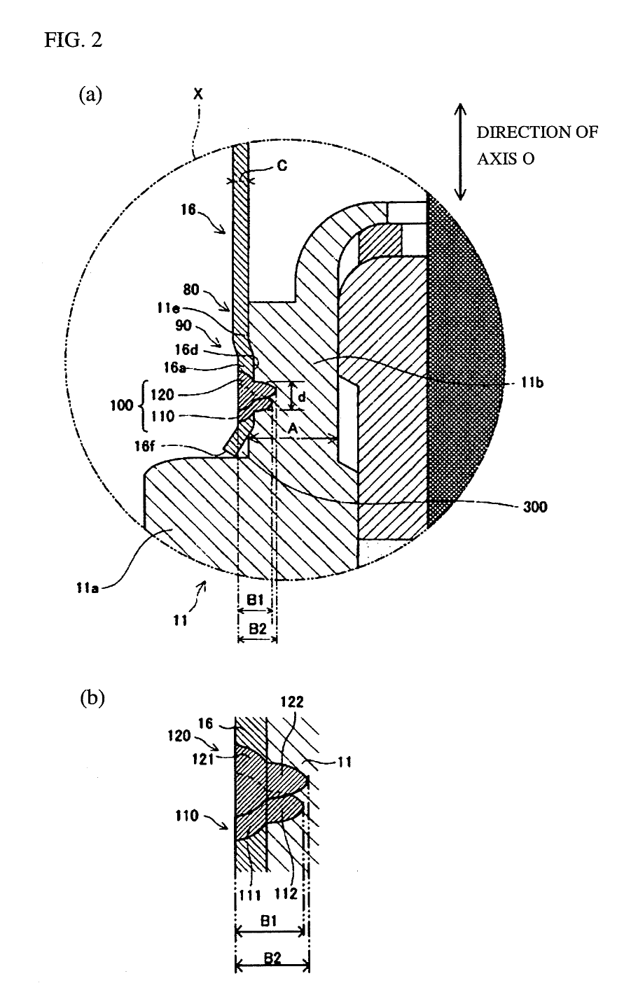

FIG. 2 is a schematic section view showing the detailed configuration of the weld joint 100 between the metal shell 11 and the outer metal casing 16 according to the first embodiment, where FIG. 2(a) shows the overall configuration of the weld joint 100; and FIG. 2(b) schematically shows a front weld zone 110 and a rear weld zone 120 of the weld joint 100. The cross section view of FIG. 2(a) is an enlarged drawing of a circled area X of FIG. 1. As shown in FIG. 2(a), the metal shell 11 is inserted in the outer metal casing 16 to define an overlap area 80 in which an outer circumferential surface 11e of the cylindrical portion 11b of the metal shell 11 faces an inner circumferential surface 16d of the front end portion 16a of the outer metal casing 16. The overlap area 80 is crimped throughout its entire circumference from outside. The resulting ring-shaped crimped area is hereinafter called a crimped area 90. Then, the metal shell 11 and the outer metal casing 16 are laser welded together via the weld joint 100 by irradiating a laser beam onto the entire circumference of the crimped area 90 in a circumferential direction of the outer metal casing 16. In the first embodiment, this laser welding operation is performed at a plurality of positions (two positions in the first embodiment) displaced from each other in the direction of the axis O so that the weld joint 100 is formed with a front weld zone 110 and a rear weld zone 120 as shown in FIG. 2(a).

As shown in FIG. 2(b), the front weld zone 110 has an outer weld region 111 formed and located in the front end portion 16a of the outer metal casing 16 and an inner weld region 112 formed and located in the cylindrical portion 11b of the metal shell 11. Similarly, the rear weld zone 120 has an outer weld region 121 formed and located in the front end portion 16a of the outer metal casing 16 and an inner weld region 122 formed and located in the cylindrical portion 11b of the metal shell 11. The weld joint 100 is formed by repeatedly performing the laser welding operation in such a manner that the inner weld regions 112 and 122 of the front and rear weld zones 110 and 120 partially overlap each other as shown in FIG. 2(a). The laser welding operation can be performed with the irradiation of e.g. a known YAG laser beam. The irradiation of the laser beam causes melting of a part from the front end portion 16a to the cylindrical portion 11b and allows joining of the overlap area 80 (the front end portion 16a and the cylindrical portion 11b) by the formation of the front and rear weld zones 110 and 120 astride a boundary between the front end portion 16a and the cylindrical portion 11b.

When the front end portion 16a of the outer metal casing 16 is crimped onto the cylindrical portion 11b of the metal shell 11, there may be a gap 300 left between a front edge 16f of the outer metal casing 16 and the cylindrical portion 11b of the metal shell 11. If the gas sensor 10 gets wet during its use, water penetrates into the gap 300 by its capillary action. As the water is unlikely to be volatized from such a closed gap 300, the front and rear weld zones 110 and 120 might be kept in contact with the residual water in the gap 300 for a long term. The front and rear weld zones 110 and 120, which have been once molten by the laser welding operation, are relatively susceptible to corrosion by water. Thus, corrosion proceeds at interfaces between the front and rear weld zones 110 and 120 and non-weld zones during the long-term contact of water with these weld zones 110 and 120. This results in a deterioration of the joint strength between the metal shell 11 and the outer metal casing 16. Especially when the gas sensor 10 is mounted for use on a vehicle, calcium chloride (CaCl₂) of a road snow removal agent may be mixed into the residual water within the gap 300 so that the resulting salt water promotes the corrosion of the weld zones.

As the corrosion starts from the gap 300 and progresses from the lower side to the upper side of the drawing along the direction of the axis O, it is feasible by increasing the weld width d to prevent the joint strength of the metal shell 11 and the outer metal casing 16 from being deteriorated due to such corrosion. In the present description, the term “weld width d” refers to a width of the weld joint in the direction of the axis O at the boundary between the outer metal casing 16 and the metal shell 11 (i.e. at the contact face between outer circumferential surface 11e of the metal shell 11 and the inner circumferential surface 16d of the outer metal casing 16). The laser welding operation is performed twice at positions displaced from each other in the direction of the axis O, i.e., in the direction of progress of the corrosion so as to form two adjacent weld zones (front and rear weld zones 110 and 120) in the first embodiment, whereby the weld width d can be made wider than that formed by one laser welding operation.

As mentioned above, the weld joint 100 has front end rear weld zones 110 and 120 formed at positions axially displaced from each other on the overlap area 80 between the front end portion 16a of the outer metal casing 16 and the cylindrical portion 11b of the metal shell 11. It is therefore possible to increase the weld width d i.e. the total width of the front and rear weld zones 110 and 120 and possible to retard of the progress of the corrosions of the front and rear weld zones 110 and 120 and, by extension, to retard the entry of water into the gas sensor 10.

Further, the front end rear weld zones 110 and 120 are formed in such a manner that the rear weld zone 120 partially overlays the front weld zone 110 as shown in FIG. 2(b). This is apparent from the fact that: a border of the rear weld zone 120 is visually recognizable as indicated by a solid line; and a border of the front weld zone 110 is not visually recognizable as indicated by a dotted line as in FIG. 2(b). It is herein noted that, in the overlap between the front and rear weld zones 110 and 120, the border of the overlaid weld zone (in the first embodiment, the border of the front weld zone 110) is indicated by the dotted line for purposes of illustration.

When the rear weld zone 120 partially overlays the front weld zone 110, the laser welding operation has been repeatedly preformed by displacing the position of the laser irradiation from the front side to the rear side of the overlap area 80. It is thus possible to prevent the gap 300 between the outer casing 16 and the metal shell 11 from being increased during the laser welding and, as a result, possible to not only avoid the entry of water into the gas sensor 10 but also attain the desired form of the weld zone 110, 120.

In the gas sensor 10 of the first embodiment, the inner weld regions 112 and 122 of the front and rear weld zones 110 and 120 partially overlap each other. It is possible to increase the welding strength of the outer metal casing 16 and the metal shell 11 by the formation of such an overlap between the inner weld regions 112 and 122.

Furthermore, the front and rear weld zones 110 and 120 are formed by adjusting the laser output during the laser welding operation and thereby controlling a thickness (welding depth) B1 of the front weld zone 110 and a thickness (welding depth) B2 of the rear weld zone 120 in such a manner that each of the thickness B1 and the thickness B2 is at least greater than a thickness C of the outer metal casing 16 (for examples, twice or greater than the thickness of the outer metal casing 16) and in such a manner that each of the front and rear weld zones 110 and 120 extends to a point not more than approximately half of a radial thickness A of the metal shell 11 from the outer circumferential surface of the metal shell 11. It is possible to cause melting and mixing of components of the outer metal casing 16 and the metal shell 11 assuredly and increase the welding strength of the outer metal casing 16 and the metal shell 11 by controlling the thickness of each of the front and rear weld zones 110 and 120 as mentioned above.

In the first embodiment, the thickness B2 of the rear weld zone 120 is also set greater than the thickness B1 of the front weld zone 110. Even though the front weld zone 110 is formed in a state that the metal shell 11 and the outer casing 16 have not yet been joined together, it is possible to attain the desired form of the front weld zone 110 by setting the depth B1 of the front weld zone 110 slightly smaller than the target depth. It is also possible to attain the desired form of the rear weld zone 120 by setting the depth B2 of the rear weld zone 120 greater than the depth B1 of the front weld zone 110.

Although the laser welding operation is performed twice in the first embodiment, the number of times the laser welding operation is performed is not limited to two and may be two or more. The laser welding operation can be performed a plurality of times as appropriate depending on the thickness and material of each of the metal shell 11 and the outer metal casing 16.

A3. Manufacturing Method

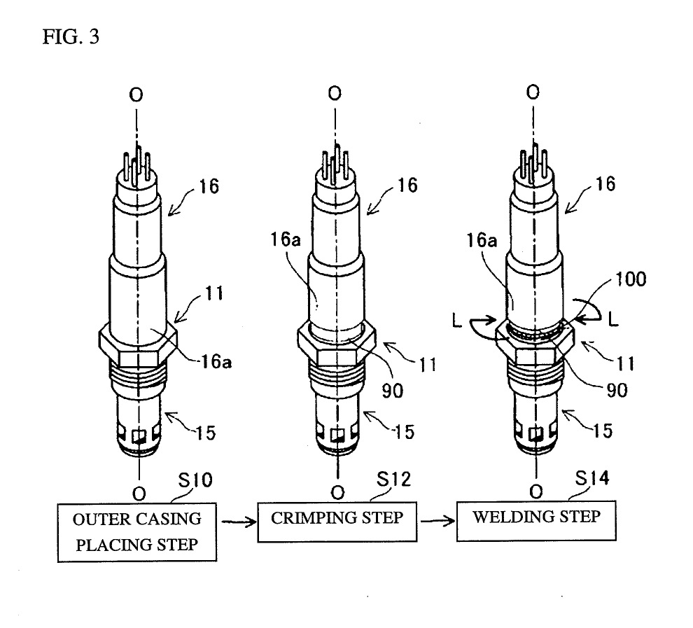

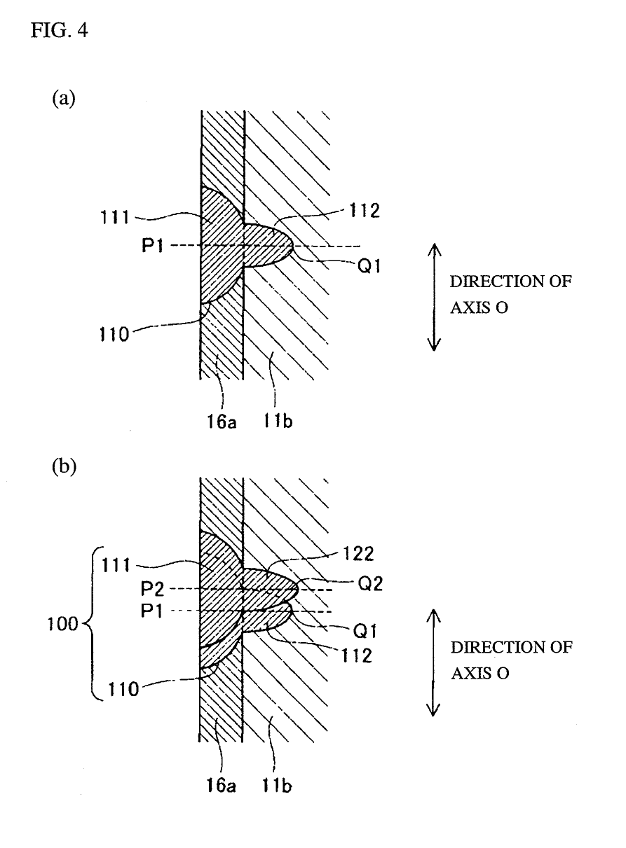

A gas sensor manufacturing method will be next explained below with reference to FIGS. 3 and 4. FIG. 3 is a flowchart of process steps for manufacturing the gas sensor 10 according to the first embodiment. FIG. 4 is a schematic view showing a welding step during the manufacturing of the gas sensor 10 according to the first embodiment. It is herein noted that the explanation of FIG. 3 will be focused on the joining of the metal shell 11 and the outer metal casing 16 of the gas sensor 10; and the detained explanations of the process steps of the other structural members of the gas sensor 10 will be omitted as those process steps can be carried out in known manners.

The metal shell 11 and the outer metal casing 16 are prepared and assembled with the other structural members by any known processes. This assembling step includes placement of the oxygen sensor element 20 in the metal shell 11 and placement of the separator 18 in the outer metal casing 16. First, the protector 15 is joined by welding etc. to the metal shell 11. The oxygen sensor element 20 is inserted in the though hole 58 of the metal shell 11 after arrangement of the insulator 13 in the metal shell 11. Subsequently, the talc 14 and the sleeve 13b are inserted in the through hole 58 of the metal shell 11. The metal shell 11 is crimped, thereby compressing the talc 14 in such a manner that the talc 14 fills in clearance between the metal shell 11 and the oxygen sensor element 20 to retain the oxygen sensor element 20 in the metal shell 11. The sensor output leads 19 and 19b are connected to the inner and outer terminal members 30 and 40, respectively, and then, passed through the separator 18 and the grommet 17. Further, the heater leads 12b and 12c are connected to the ceramic heater 50 and passed through the separator 18 and the grommet 17. After that, the separator 18 and the grommet 17 are fitted in the outer metal casing 16.

The outer metal shell 16 is next placed in position on the metal shell 11 (step S10). More specifically, the outer metal casing 16 is fitted on the rear end portion of the metal shell 11 so as to accommodate the rear end portion of the oxygen sensor element 20 in the outer metal casing 16 and to arrange the ceramic heater 50 in the inner space G of the oxygen sensor element 20. By this, the outer metal shell 16 is placed in such a manner that the inner circumferential surface 16d of the front end portion 16a of the outer metal casing 16 faces the outer circumferential surface 11e of the cylindrical portion 11b of the metal shell 11 (see FIG. 2).

The front end portion 16a of the outer metal casing 16 is then crimped throughout its circumference (eight-directional round crimping) (step S12). In this crimping step, the crimped area 90 is formed for temporary fixing of the outer metal casing 16 and the metal shell 11. The separator 18 and the grommet 17 are further fixed by crimping the outer metal casing 16 onto the separator 18 and the grommet 17 (eight-directional round crimping).

The metal shell 11 and the outer metal casing 16 are joined together by laser welding the entire circumference of the crimped area 90 in the circumferential direction of the outer metal casing 16 as indicated by an arrow L (step S14). In the first embodiment, the laser welding operation is performed twice so that the front and rear weld zones 110 and 120 are formed at positions displaced from each other in the direction of the axis O.

The laser welding step of the first embodiment will be explained in more detail below with reference to FIG. 4. FIG. 4(a) and FIG. 4(b) show the first and second laser welding operations, respectively. As shown in FIG. 4(a), the first laser welding operation is carried out to form the front weld zone 110 in such a manner that an apex Q1 of the inner weld region 112 of the front weld zone 110 is located at horizontally the same position as a position P1 of the direction of the axis O. As shown in FIG. 4(b), the second laser welding operation is then carried out by shifting the position of laser welding equipment relative to the gas sensor 10 in the direction of the axis O, to thereby form the rear weld zone 120 in such a manner that an apex Q2 of the inner weld region 122 of the rear weld zone 120 is located at horizontally the same position as a position P2 of the direction of the axis O, which is slightly displaced to the rear from the position P1 in the direction of the axis O. At this time, the relative position of the laser welding equipment and the gas sensor 10 is controlled in such a manner that the inner weld region 112 of the front weld zone 110 formed by the first laser welding operation and the inner weld region 122 of the rear weld zone 120 formed by the second laser welding operation partially overlap each other.

Further, the laser beam is irradiated by the laser welding equipment from the direction subsequently perpendicular to the direction of the axis O so that the outer metal casing 16 and the metal shell 11 are molten at substantially the same degree. In the above laser welding step, the metal shell 11 and the outer metal casing 16 are joined together by the formation of the front and rear weld zones 110 and 120 astride the boundary between the metal shell 11 and the outer metal casing 16. In this way, the gas sensor 10 is completed.

In the manufacturing method of the gas sensor 10 of the first embodiment, the laser welding operation is performed a plurality of times at positions displaced from each other in the direction of the axis O on the overlap area 80 between the front end portion 16a of the outer metal casing 16 and the cylindrical portion 11b of the metal shell 11 as mentioned above. As the thus-formed front and rear weld zones 110 and 120 can secure a wider weld width (see FIG. 2), it is possible to retard the progress of corrosion of the weld joint and, by extension, possible to retard the entry of water into the gas sensor 10.

Further, the laser welding operation is repeatedly performed in such a manner that the inner weld regions 112 and 122 of the front and rear weld zones 110 and 120 partially overlap each other. It is possible to increase the welding strength of the outer metal casing 16 and the metal shell by the formation of such an overlap between the inner weld regions.

Furthermore, the laser welding operation is repeatedly performed by displacing the position of the laser welding operation from the front side to the rear side of the overlap area 80 in the direction of the axis O. It is thus possible to prevent the gap 300 between the outer casing 16 and the metal shell 11 from being increased during the welding step and, as a result, possible to not only avoid the entry of water into the gas sensor 10 but also attain the desired form of the weld zone 110, 120.

In the manufacturing method of the gas sensor 10 of the first embodiment, the laser welding operation is preferably performed in such a manner that the depth of the rear weld zone 120 (second weld zone) formed by the second laser welding operation becomes greater than the depth of the front weld zone 110 (first weld zone) formed by the first laser welding operation. In the thus-formed weld joint 100, the thickness B2 of the rear weld zone 120 is made greater than the thickness B1 of the front weld zone 110. Even though the first weld zone 110 is formed in a state that the metal shell 11 and the outer casing 16 have not yet been joined together, it is possible to attain the desired form of the front weld zone 110 by setting the depth B1 of the front weld zone 110 slightly smaller than the target depth. It is also possible to attain the desired form of the rear weld zone 120 by setting the depth B2 of the rear weld zone 120 greater than the depth B1 of the front weld zone 110.

It is herein noted that, in the first embodiment, the oxygen sensor element 20 corresponds to the claimed sensor element; and the outer metal casing 16 corresponds to the claimed outer casing.

### B. Modification Examples

Although the laser welding step is carried out in such a manner that the front and rear weld zones 110 and 120 partially overlap each other in the first embodiment, the front and rear weld zones 110 and 120 may not overlap each other. It may alternatively be feasible to form the rear weld zone 120 by the first laser welding operation and then form the front weld zone 110 by the second laser welding operation although the laser welding step is carried out to form the front weld zone 110 by the first laser welding operation and then form the rear weld zone 120 by the second laser welding operation in the first embodiment. Although the thickness B2 of the rear weld zone 120 formed by the second laser welding operation is set greater than the thickness B1 of the front weld zone 110 formed by the first laser welding operation in the first embodiment, the thickness of the front weld zone 110 and the thickness of the rear weld zone 120 may be set substantially equal to each other.

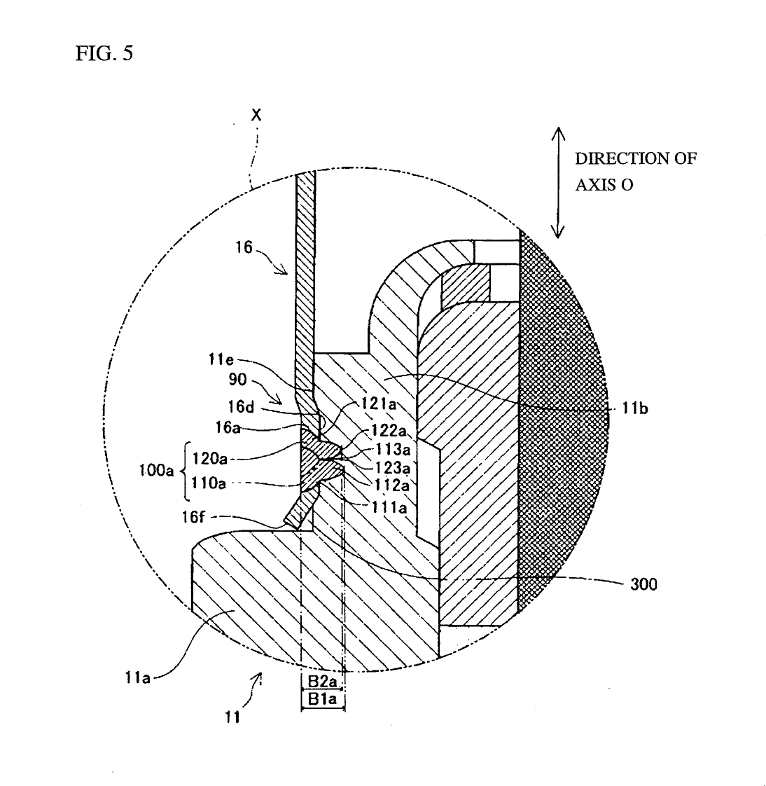

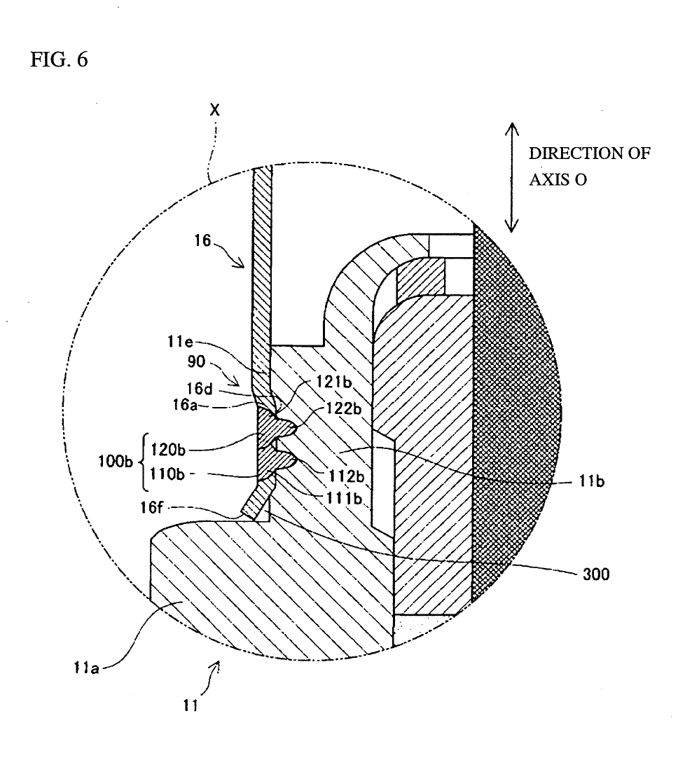

Modification examples of the front and rear weld zones 110 and 120 will be explained below with reference to FIGS. 5 and 6. FIGS. 5 and 6 are schematic section views showing the detailed configurations of weld joints between the metal shell 11 and the outer metal casing 16 according to modifications of the first embodiment. As in the case of FIG. 2, each of the cross section views of FIGS. 5 and 6 is an enlarged drawing of the vicinity of the weld joint. In the modification examples of FIGS. 5 and 6, the same parts and portions are designated by the same reference numerals as those of the first embodiment.

The first modification example of FIG. 5 will be now explained below. As shown in FIG. 5, the metal shell 11 and the outer metal casing 16 are laser welded together via a weld joint 100a by irradiating a laser beam onto the entire circumference of the crimped area 90 in a circumferential direction of the outer metal casing 16. This laser welding operation is performed twice at positions displaced from each other in the direction of the axis O, as in the case of the first embodiment, so as to form the weld joint 100a with front and rear weld zones 110a and 120a. In the first modification example, however, the laser welding operation is repeatedly performed to form the rear weld zone 120a by the first laser welding operation and to form the front weld zone 110a by the second laser welding operation at a position slightly displaced to the front from that of the first laser welding operation in the direction of the axis O. By this, the front and rear weld zones 110a and 120a are formed in such a manner that the front weld zone 110a partially overlays the rear weld zone 120a. This is apparent from the fact that a border of the front weld zone 110a is visually recognizable as indicated by a solid line; and a border of the rear weld zone 120a is not visually recognizable as indicated by a dotted line as in FIG. 5. The front and rear weld zones 110a and 120a have outer weld regions 111a and 121a formed and located in the front end portion of the outer metal casing 16 and inner weld regions 112a and 122a formed and located in the cylindrical portion of the metal shell 11, respectively. The inner weld regions 112a and 122a of the front and rear weld zones 110a and 120a are located adjacent to each other. Namely, an interface 113a between the inner and outer weld regions 112a and 111a of the weld zone 110a and an interface between the inner and outer weld regions 122a and 121a of the weld zone 120a abut each other.

Further, a depth B1a of the front weld zone 110a (second weld zone) formed by the second laser welding operation is made greater than a depth B2a of the rear weld zone 120a (first weld zone) formed by the first laser welding operation as shown in FIG. 5. Even though the rear weld zone 120a is formed in a state that the metal shell 11 and the outer casing 16 have not yet joined together, it is possible to attain the desired form of the rear weld zone 120 by setting the depth B2a of the rear weld zone 120a slightly smaller than the target depth. It is also possible to attain the desired form of the front weld zone 110a by setting the depth B1a of the front weld zone 110a greater than the depth B2a of the rear weld zone 120a.

Next, the second modification example of FIG. 6 will be explained below. As shown in FIG. 6, the metal shell 11 and the outer metal casing 16 are laser welded together via a weld joint 100b by irradiating a laser beam onto the entire circumference of the crimped area 90 in a circumferential direction of the outer metal casing 16. This laser welding operation is performed twice at positions displaced from each other in the direction of the axis O, as in the case of the first embodiment, so as to form the weld joint 100b with front and rear weld zones 110b and 120b. In the second modification example, however, a thickness B1b of the front weld zone 110b and a thickness B2b of the rear weld zone 120b are set substantially equal to each other as shown in FIG. 6 in contrast to the first embodiment. The front and rear weld zones 110b and 120b have outer weld regions 111b and 121b formed and located in the front end portion of the outer metal casing 16 and inner weld regions 112b and 122b formed and located in the cylindrical portion of the metal shell 11, respectively. The inner weld regions 112b and 122b of the front and rear weld zones 110b and 120b are located apart from each other in the direction of the axis O. Namely, the inner weld regions 112b and 122b do not overlap each other. On the other hand, the outer weld regions 111b and 121b partially overlap each other in FIG. 6 although these outer weld regions 111b and 121b do not necessarily overlap each other.

In the first and second modification examples of FIGS. 5 and 6, the laser welding operation is repeatedly performed as mentioned above so that the overall weld width (i.e. the sum of the weld width of the front weld zone 110a, 110b and the weld width of the rear weld zone 120a, 120b) can be made wider than that formed by one laser welding operation. It is therefore possible in these modification examples to retard the entry of water into the gas sensor 10 as in the case of the first embodiment.

Although the present invention has been described with reference to the above embodiments, the present invention is not limited to these specific exemplary embodiments. Various modifications and variations of the embodiments described above will occur without departing from the scope of the present invention. For example, a rectangular plate-shaped sensor element may be used although the oxygen sensor element 20 is formed into a bottomed cylindrical shape in the first embodiment and in the modification examples. The present invention can be applied to either a NOx sensor, a H₂ sensor, a temperature sensor or the like although the first embodiment and the modification examples each refers to the oxygen sensor 10.

### DESCRIPTION OF REFERENCE NUMERALS

10: Gas sensor
11: Metal shell
12b: Heater lead
13: Insulator
13b: Sleeve
14: Talc
15: Protector
15a: Outer protector member
15b: Inner protector member
16: Outer metal casing
17: Grommet
18: Separator
19, 19b: Sensor output lead
20: Oxygen sensor element
30: Inner terminal member
40: Outer terminal member
50: Ceramic heater
85: Filter
86: Metal pipe
90: Crimped portion
100, 100a, 100b: Weld joint
110, 110a, 110b: Front weld zone
120, 120a, 120b: Rear weld zone
111, 111a, 111b: Outer weld region
112, 112a, 112b: Inner weld region
113a, 123a: Interface
121, 121a, 121b: Outer weld region
122, 122a, 122b: Inner weld region
300: Gap

1. A manufacturing method of a gas sensor, the gas sensor comprising: a sensor element extending in an axis direction of the gas sensor and having at a front end portion thereof a sensing section to detect a measurement gas; a metal shell having a cylindrical portion to surround an outer circumference of the sensor element, with the front end portion and a rear end portion of the sensor element being exposed to an outside of the metal shell; and a cylindrical outer casing fixed to the metal shell to surround the rear end portion of the sensor element, the manufacturing method comprising:
an outer casing placing step for placing the outer casing on the metal shell in such a manner that a front end portion of the outer casing surrounds the cylindrical portion of the metal shell; and
a welding step for performing a laser welding operation on the entire circumference of an overlap area between the front end portion of the outer casing and the cylindrical portion of the metal shell and thereby forming a weld zone astride a boundary between the front end portion of the outer casing and the cylindrical portion of the metal shell,
wherein, in the welding step, the laser welding operation is performed a plurality of times at positions displaced from each other in the axis direction of the gas sensor.

2. The manufacturing method of the gas sensor according to claim 1, wherein the laser welding operation is performed the plurality of times to form a plurality of weld zones in the welding step in such a manner that adjacent two of the weld zones have inner weld regions located in the cylindrical portion of the metal shell and partially overlapping each other.

3. The manufacturing method of the gas sensor according to claim 1, wherein the laser welding operation is performed the plurality of times by displacing the position of the laser welding operation from a front side to a rear side of the overlap area in the welding step.

4. The manufacturing method of the gas sensor according to claim 1, wherein the welding step is carried out to form a first weld zone by the first laser welding operation and to form a second weld zone by the second or subsequent laser welding operation in such a manner that the second weld zone is greater in depth than the first weld zone.

5. A gas sensor, comprising:
a sensor element extending in an axis direction of the gas sensor and having at a front end portion thereof a sensing section to detect a measurement gas;
a metal shell having a cylindrical portion to surround an outer circumference of the sensor element, with the front end portion and a rear end portion of the sensor element being exposed to an outside of the metal shell;
a cylindrical outer casing fixed to the metal shell to surround the rear end portion of the sensor element; and
a plurality of weld zones each formed astride a boundary between a front end portion of the outer casing and the cylindrical portion of the metal shell throughout the entire circumference of an overlap area between the front end portion of the outer casing and the cylindrical portion of the metal shell and displaced in position from each other in the axis direction of the gas sensor.

6. The gas sensor according to claim 5, wherein adjacent two of the weld zones partially overlap each other in such a manner that, when viewed in cross section along the axis, a rear one of the adjacent two of the weld zones partially overlays a front one of the adjacent two of the weld zones.

7. The gas sensor according to claim 6, wherein the rear one of the adjacent two of the weld zones is greater in depth than the front one of the adjacent two of the weld zones.

8. The gas sensor according to claim 5, wherein adjacent two of the weld zones have inner weld regions located in the cylindrical portion of the metal shell and partially overlapping each other.

9. The gas sensor according to claim 5, wherein the weld zones includes a first weld zone and a second weld zone partially overlaying the first weld zone and having a depth greater than a width of the first weld zone.

10. The gas sensor according to claim 5, wherein the weld zones extend to a point not more than half of a thickness of the cylindrical portion of the metal shell.
